(19) United States
(12) Patent Application Publication
(10) Pub. No.: US 2001/0033858 A1
(43) Pub. Date: Oct. 25, 2001

(54) Transdermal drug patch

(76) Inventor: Jie Zhang, Salt Lake City, UT (US)

Correspondence Address: Michael F. Krieger, Kirton & McConkie, 1800 Eagle Gate Tower, 60 East South Temple, Salt Lake City, UT, 84111, US

(21) Appl. No.: 09/796,200

(22) Filed: Feb. 28, 2001

Related U.S. Application Data
(60) Provisional application No. 60/185,893, filed on Feb. 29, 2000.

(51) Int. Cl. A61K 9/70

(52) U.S. Cl. 424/443

(57) ABSTRACT

The present invention is directed toward a formulation for supplying additional drug for delivery in a transdermal drug delivery device. The invention comprises a drug, such as fentanyl that is capable of transdermal delivery, and a solution having a pre-designed solubility for the drug. The solution dissolves only a portion of said drug and allows a significant portion of the drug to remain undissolved in solution, thus providing extra drug to be delivered at a consistent, controlled delivery rate. The invention may used in conjunction with controlled heat.

### RELATED APPLICATIONS

This application claims priority to U.S. Provisional Application No. 60/185,893 filed Feb. 29, 2000.

### BACKGROUND OF THE INVENTION

1. The Field of the Invention

The present invention is directed toward an improved transdermal drug delivery patch. More specifically, the present invention is directed toward improving drug deliver patches for use with temperature modification devices.

2. Present State of the Art

Transdermal drug delivery to administer drugs to patients is an effective and efficient method for delivering certain drugs to patients. Transdermal drug delivery is convenient, noninvasive, and in some cases provides a more effective method for delivering a drug. However, transdermal drug delivery patches have a number of limitations and disadvantages.

Typically when a drug patch is applied to a patient's skin, the drug in the drug formulation is absorbed into the patient's skin. The absorption rate at which the drug leaves the drug formulation and penetrates across the patient's skin is dependent upon a number of factors including the formulation of the drug. As the drug enters the patient's body, the drug concentration in the drug formulation decreases and the drug concentration in the patient's skin and surrounding tissues increases. Thus, as the drug is being used from a formulation in which all drug is dissolved, the decreasing drug concentration results in the decrease in the overall absorption rate of the drug into the patient's body.

Once past the patient's skin, some of the drug is absorbed into the patient's systemic circulation and carried throughout the body to a desired target tissue and some gets stored in tissues and released slowly into the systemic circulation (depot effect). The concentration of the drug in the patient's systemic circulation (the blood drug concentration) will be dependent upon the transdermal permeation rate of the drug and release rate from depot into the systemic circulation.

The ease with which drugs can be delivered through the skin has made the use of transdermal drug delivery patches popular for certain drugs. A number of patched are available for delivering a variety of drugs. Androderm patches manufactured by TheraTech (now Watson Pharmaceutical), Testosterone and analgesic patches as manufactured by Alza, and nicotine scopolamine patches are also available. Other types of transdermal drug delivery patches are also known in the art.

If all the drug in the formulation is in dissolved state, the permeation driving force will decrease over time as the drug is depleted from the formulation. Theoretically the delivery rate will decrease over time. However, this is a very slow process since transdermal drug delivery rate is usually quite low, and the decreasing driving force may be compensated by the depot effect. For example, the 25 μg/hr Duragesic fentanyl transdermal patch contains 2.5 mg of fentanyl and is intended to deliver 25 μg of it into the body per hour. That is a rate of 1% per hour. The patch is designed to be used for 72 hours. Theoretically, at the beginning of the 3^{rd} day, the permeation driving force is reduced by about half due to the 48% depletion of the fentanyl in the formulation. Indeed, it is observed that some patients complain of less than satisfactory pain control on the third day. However, fentanyl has a significant depot effect, and the decrease in the transdermal delivery rate is probably compensated somewhat by the release from the depot. That may be the reason why there are not more complaints about poor pain control on the third day. Therefore, the decreasing delivery rates have not been a major problem for Duragesic patch.

Some of our pending patent applications are related to the use of heat to shorten the onset time of transdermal fentanyl patches and/or to provide rapid delivery of extra fentanyl using controlled heat on a transdermal fentanyl patch to treat breakthrough pain. The rationale is that heat can increase skin permeability and drive fentanyl in depot tissues into the blood circulation. As can be seen in Example 3, heating the fentanyl patch in the early phase of the application significantly speeds up the increase in serum fentanyl concentrations and thus shortens the time to reach steady state concentrations. Example 3 also reveled that heating the fentanyl patch after steady state serum fentanyl concentrations are reached can rapidly and significantly increase the serum level. That is because heat can release fentanyl stored in depot tissues into the systemic circulation. However, since all the extra fentanyl is ultimately from the formulation in the patch, these heating manipulations will greatly deplete additional amounts of fentanyl from the patch. If all fentanyl in the transdermal formulation is dissolved in the formulation, these heating manipulations will cause extra decrease in the concentration of dissolved fentanyl in the formulation. Since the passive transdermal permeation driving force of a drug is usually proportional to the concentration of dissolved drug in the formulation, the extra decrease in dissolved fentanyl formulation may result in undesirably low delivery rates in the later phase of the application. After those heating manipulations, the depot effect may not be able to compensate the loss of permeation driving force because the decrease in permeation driving force might be too much and because the depot itself is at least partially depleted in the heating manipulations. In addition, some patients may use much more extra drug by the heating manipulations than others. The difference in heat-induced depletion in the early phase of the application will then result in different concentrations of fentanyl in the formulation and hence different delivery rates in the later phase of the application, which is very undesirable.

One might think putting more fentanyl in the formulation can solve the problem. However, simply increasing the fentanyl concentration in the formulation may not be a good solution, because that may create too high delivery rates in the early phase of the application. In addition, it still does not solve the problem of different delivery rates between patients who have and have not performed the heating manipulations.

Therefore, although the heating manipulations discussed above can be very beneficial, it poses a challenge in the formulation design.

It would therefore be advantageous to develop a dermal drug delivery system which provides consistent drug delivery rates for substantially a portion of or for the entire application life. It would therefore be advantageous to provide a formulation for transdermal drug delivery that provides a constant delivery rate regardless of previous delivery amount. It would also be advantageous to provide a dermal drug delivery system with a longer duration for consistent drug delivery. Additionally, it would be advantageous to provide a drug delivery patch which provides consistent drug delivery rates even when extra drugs are depleted from the patch by heating to provide quicker onset of effect or to provide extra drug to accommodate changing needs. More specifically, it would be advantageous to develop a transdermal delivery system for fentanyl or other potent analgesic drugs that can provide consistent delivery rates over long period of time even if extra drug is depleted from the system or from the depot created by the system to provide other benefits.

The present invention provides a method and apparatus for improving the transdermal drug delivery.

### OBJECTS AND BRIEF SUMMARY OF THE INVENTION

The present invention provides a means for automatically supplying additional dissolvable drug to a formulation in a dermal drug delivery system. The formulation of the present invention provides a secondary drug supply which replenishes the drug in solution as the drug in solution is delivered into the patient's body. The secondary supply is not directly available for transdermal permeation, but can keep the concentration of the drug in solution at a constant, saturated level. The formulation of the present invention has both dissolved and undissolved drug particles and a pre-designed solubility for the drug. As the dissolved drug enters the patient's body, enough undissolved drug particles become dissolved so that the concentration of the dissolved drug is kept at a constant level. The key in this invention is to select a formulation in a transdermal drug delivery system that has the drug solubility high enough to provide sufficient transdermal permeability but low enough so that significant amount of the drug can exist in the formulation as undissolved particles. More specifically, the present invention provides means for keeping the concentration of dissolved fentanyl in the formulation of a transdermal fentanyl delivery system at constant levels by selecting a solvent system that has a fentanyl solubility that allows the delivery of fentanyl transdermally at therapeutically sufficient rates but also allows significant amount of fentanyl in the formulation to exist as undissolved particles.

The present invention keeps the transdermal permeation rates at constant levels despite different amounts that might have been depleted from the patch.

### DETAILED DESCRIPTION OF THE PREFERRED EMBODIMENTS

It will be readily understood that the components of the present invention, as generally described and illustrated herein, could be arranged and designed in a wide variety of different configurations. Thus, the following more detailed description of the embodiments of the system and apparatus of the present invention is not intended to limit the scope of the invention, as claimed, but it is merely representative of the presently preferred embodiments of the invention.

### EXAMPLE 1

In one embodiment, a formulation comprising fentanyl is incorporated into a transdermal fentanyl delivery patch. The formulation comprises a solvent system that has a fentanyl base solubility of between 0.1-50 mg/mL, preferably between 0.5-20 mg/mL, and most preferably between 1-10 mg/mL. For example, the solvent system is chosen to have a solubility for fentanyl base of 5 mg/mL. One mL of the solvent system is then mixed with 15 mg of fentanyl base and other excipients such as thickening agent(s), permeation enhancer, or agent(s) that provides adhesiveness to form a formulation which has a dissolved fentanyl concentration of about 5 mg/mL and about 10 mg of undissolved fentanyl particles per mL. The formulation is then incorporated into a transdermal drug delivery patch having a skin contact area of 10 square centimeters. Assuming with the help of the permeation enhancer in the formulation, the skin permeability is 2×10⁻⁷ cm/sec. The transdermal fentanyl rate for the patch then will be:

R=P*C*A=2×10⁻⁷*5000 μg/mL*10 cm²=0.01 μg/second=36 μg/hour

Where R is the delivery rate, P is permeability coefficient in cm/sec, C is concentration of dissolved fentanyl in μg/mL and A is area of contact in cm².

At the beginning of the patch application, the patch has 5 mg fentanyl base in dissolved form and other 10 mg as undissolved particles. As dissolved fentanyl gets absorbed transdermally, undissolved fentanyl particles will dissolve into the solvent get dissolved so that the formulation keeps the dissolved fentanyl concentration at 5 mg/mL, until all undissolved particles are dissolved. The solubilization rate of undissolved fentanyl particles is the same as the transdermal absorption rate, 36 mcg/hour. Therefore, the 10 mg undissolved fentanyl particles will take 10,000 μcg/36 mcg=278 hours to dissolve. In other words, the patch can keep a constant delivery rate for more than 10 days. Even if 5 mg of extra fentanyl is depleted from the patch by heating manipulations to treat breakthrough pain, the patch can still provide constant delivery rate for more than 5 days.

There are two ways to select a solvent system with desired fentanyl solubility. One way to select a solvent system is by using a proper pH buffer system. Fentanyl solubility in aqueous solution or gel strongly depends on the pH of the medium. The solubility is much higher at low pH than at high pH. Therefore, selecting a proper pH should enable one to obtain a desired solubility. In addition, a pH buffer system usually also has the ability to maintain the pH against solvent loss (i.e. water evaporation). Therefore, the use of pH buffer system may also provide stability in solubility against solvent evaporation. Since pH buffer systems usually only work in aqueous solutions, the formulation should at least contain some water.

Another way to select a solvent system is by selecting good solvent(s) and non-solvent(s) and the mixture of right ratio of them. For example, fentanyl base has high solubility in alcohol and low solubility in water. One should be able to obtain desired solubility in alcohol-water mixture by selecting the right alcohol-to-water ratio.

A mixture of good solvent-poor solvent (where the mixture does not have volatile component) is desirable if the formulation is to be used in a matrix patch. The formulation containing the drug is also used as an adhesive for affixing the patch on the skin.

### EXAMPLE 2

A transdermal nicotine system in combination with controlled heat may be used to alleviate baseline craving and episodes of breakthrough craving. Placing a heating patch on top of the nicotine patch when an episode of breakthrough craving occurs delivers more nicotine into the systemic circulation. The heating duration of the heating patch is preferably designed to be long enough to deliver sufficient extra nicotine. The patient may remove the heating patch when the breakthrough craving begins to diminish. Thus, using controlled heat, the nicotine patch can alleviate both baseline craving and episodes of breakthrough craving. However, the increased delivery of nicotine by the heat may result in a sharp drop in the concentration of nicotine in the formulation, resulting in a slower and variable delivery rate when the heating is discontinued.

By employing the present invention in a transdermal nicotine system, such as a nicotine patch with a formulation having dissolved and undissolved nicotine, the concentration of dissolved nicotine in the formulation is kept at desired and constant levels. Thus, breakthrough craving can be treated using heat without causing a dramatic decline or change in the concentration of dissolved drug in the formulation afterwards.

### EXAMPLE 3

In another example, a patient requires a therapeutic serum fentanyl concentration that is very high in order to treat baseline pain. The required dose for the patient is high enough that inadvertent overdosing would have serious side effects such as respiratory depression. Delivery of the required dose must be precise. To maintain the required steady state, the drug delivery must be predictable and consistent and not exceed safe levels of administration.

The patient is treated with a transdermal fentanyl patch employing the formulation of the present invention. After the patch is applied, the patient's serum fentanyl concentration begins to rise, approaching, but not exceeding the therapeutic serum fentanyl concentration. As the dissolved drug leaves the formulation and enters the blood stream, the undissolved drug dissolves into the formulation, maintaining the concentration of the dissolved drug in the formulation and ensuring the serum fentanyl concentration is consistent and does not exceed safe levels of administration.

In this example “the clamped” fentanyl delivery rate provided by the fixed solubility helps minimize the variability in the delivery rates which improves patient safety.

### EXAMPLE 4

In this example, a user needs to apply a transdermal drug patch employing the formulation of the present invention for an extended period of time without the serum drug concentration dropping below a desired level. After the patch is applied, the user's serum drug concentration begins to rise, approaching desired steady state. The patch is worn for an extended period of time, (e.g. 24 hours). Toward the end of the extended application, as the dissolved drug leaves the formulation and enters the blood stream. the patient continues to receive the dug at the desired delivery rate, rather than at a decreased rate because, the undissolved drug dissolves into the formulation, maintaining the concentration of the dissolved drug in the formulation.

Any transdermal drug that provides advantages from constant delivery rates, especially constant delivery rates over an extended period of time, and /or any transdermal drug that is subject to intentional fluctuations between increased or decreased delivery rates and a desired steady state may benefit from this invention. Such drugs include fentanyl, sufentanil, nicotine, nitroglycerine and hormones such as estrogen and testosterone.

What is claimed is:

1. A formulation for supplying additional drug in a transdermal drug delivery device comprising:
a drug, and
a solution having a pre-designed solubility to dissolve only a portion of said drug to provide a controlled delivery rate.

2. The formulation of claim 1, wherein said solution further comprises a first substance into which said drug can be dissolved and a second substance into which said drug has lower solubility than said first substance.

3. The formulation of claim 1, further comprising a pH buffer, said pH buffer determining the solubility of said drug in the solution.

4. The formulation of claim 1, further comprising a permeation enhancer.

5. The formulation of claim 1, further comprising a binding agent, a thickener, or an adhesive component.

6. The formulation of claim 1, wherein said drug is a potent analgesic.

7. The formulation of claim 1, wherein an undissolved portion of said drug is a secondary drug supply.

8. The formulation of claim 1, wherein said solution has a predesigned solubility for the drug.

9. The formulation of claim 1, wherein the solution has a drug solubility high enough to provide transdermal permeability of the drug at therapeutic levels.

10. The formulation of claim 1, wherein said drug is subject to extra delivery by using controlled heating.

11. The formulation of claim 1, wherein said solution has a solubility to allow the existence of undissolved drug and thereby maintain the drug at a desired concentration in the formulation.

12. The formulation of claim 1, wherein said drug is delivered using a transdermal delivery system comprising a means for bringing the formulation into contact with the skin.

13. The formulation of claim 1, wherein said formulation comprises a solvent system having a fentanyl base solubility between 0.1 to 50 mg per ml.

14. The formulation of claim 1, wherein said formulation comprises a solvent system having a fentanyl base solubility of between about 0.5 to 20 mg per ml.

15. The formulation of claim 1, wherein said formulation comprises a solvent system having a fentanyl base solubility of between about 1 to 10 mg per ml.

16. The formulation of claim 13, wherein said solvent system has a solubility for fentanyl base of about 5 mg per ml, about 1 ml of the solvent system is mixed with about 15 mg of fentanyl base to produce a dissolved fentanyl concentration of about 5 mg per ml and an undissolved fentanyl concentration of about 10 mg per ml.

17. The formulation of claim 13, wherein said solvent system further comprises excipients are selected from the group consisting of: thickening agents, permeation enhances and adhesive agents.

18. The formulation of claim 1, wherein said formulation is brought into contact with an area of skin.

19. The formulation of claim 18, wherein said skin area is about 5-50 square cm.

20. The formulation of claim 1, wherein said drug is fentanyl.

21. The formulation of claim 1, wherein said drug is sufentanil.

22. The formulation of claim 1, wherein said drug is an analgesic.

23. The formulation of claim 1, wherein said drug is nicotine.

24. The formulation of claim 1, wherein said drug is a hormone.

25. A formulation for supplying additional drug in a transdermal drug delivery device comprising:
a drug, and
a solution having a first portion of said drug dissolved in said solution and a second portion of said drug being initially undissolved in said solution, said second portion being subsequently dissolved by controlled heating.

26. The formulation of claim 25, said solution has a pre-designed solubility for said drug capable of providing a consistent delivery rate of said drug without causing overdosing.

27. The formulation of claim 25, wherein said drug is subject to extra delivery by using controlled heating.

28. The formulation of claim 25, wherein said drug is fentanyl.

29. The formulation of claim 25, wherein said drug is sufentanil.

30. The formulation of claim 25, wherein said drug is an analgesic.

31. The formulation of claim 25, wherein said drug is nicotine.

32. The formulation of claim 25, wherein said drug is a hormone.

33. The formulation of claim 25, wherein said solution further comprises a first substance into which said drug can be dissolved and a second substance into which said drug has lower solubility than said first substance.

34. The formulation of claim 25, further comprising a pH buffer, said pH buffer determining the solubility of said drug in the solution.

35. The formulation of claim 25, further comprising a permeation enhancer.

36. The formulation of claim 25, further comprising a binding agent, a thickener, or an adhesive component.

37. The formulation for providing transdermal drug delivery at a consistent rate comprising:
a drug, said drug being capable of transdermal absorption,
a solvent, said solvent having a predesigned solubility such that said drug formulation has a substantially constant concentration of dissolved drug, when excess amount of said drug is present in said formulation.

38. The formulation of claim 37, further comprising a solvent system having a desired drug solubility.

39. The formulation of claim 37, wherein said solvent system provides a desired drug solubility using a pH buffer.

40. The formulation of claim 37, wherein said pH buffer also maintains the pH against solvent loss.

41. The formulation of claim 37, wherein the use of the pH also provides stability against solvent evaporation.

42. The formulation of claim 37, wherein said solvent system contains water.

43. The formulation of claim 37, wherein said solvent system provides a desired drug solubility through a mixture of solvents with high solubility and solvents with low solubility.
